(19) United States
(12) Patent Application Publication
(10) Pub. No.: US 2023/0130449 A1
(43) Pub. Date: Apr. 27, 2023

(54) Automated Fluid Delivery System

(71) Applicant: David Fluker, Port Allen, LA (US)

(72) Inventor: David Fluker, Port Allen, LA (US)

(21) Appl. No.: 17/509,217

(22) Filed: Oct. 25, 2021

(51) Int. Cl. A01K 7/02; A01K 67/033

(52) U.S. Cl. CPC A01K 7/025; A01K 67/033; A01K 2227/706

(57) ABSTRACT

The invention is an apparatus for providing fresh and sanitary drinking water or other delivery fluids to insects. Embodiments of the invention provide this fluid on an automated and continual basis during the breeding and growing of insects. Embodiments of the invention provide a continuous supply of fresh water to insects during the entirety of the growing cycle. The current system allows for an automated supply of water to be fed into the system on a continual basis. The current system also provides for the continuous cleaning and sanitation of the system to insure continued sanitary operation and suitability of the fluid.

### CROSS-REFERENCE TO RELATED APPLICATIONS

Not applicable.

### REFERENCE TO GOVERNMENT FUNDING SOURCES

Not applicable.

### REFERENCE TO SEQUENCE LISTING

Not applicable.

### FIELDS OF THE INVENTION

The disclosure as detailed herein is in the technical field of Animal Raising Watering and Feeding Systems. More specifically, the present disclosure relates to the technical field of Insect Watering and Feeding System.

### DESCRIPTION OF RELATED ART

Current methods involve manually placing watering devices in each pod.

### GENERAL SUMMARY OF THE INVENTION

Providing water to insects during the breeding and growing process. The current systems require that the watering pods are frequently changed and monitored. In addition, other systems do not allow for a fresh supply of water to be fed into the system. The current system allows for an automated supply of water to be fed into the system on a continual basis.

### DESCRIPTION OF FIGURES

FIG. 1 is a perspective view, which shows a Perspective Overall view of the System.

FIG. 2 is a perspective view, which shows an Overview of the Entire System Including the Fluid Sanitation System.

FIG. 3 is a perspective view, which shows a Perspective View of the Fluid Flow Restriction System.

FIG. 4 is a perspective view, which shows a Perspective View of the Continuous Fluid Dispenser.

FIG. 5 is a perspective view, which shows an Isometric View Inside of the Delivery Destination.

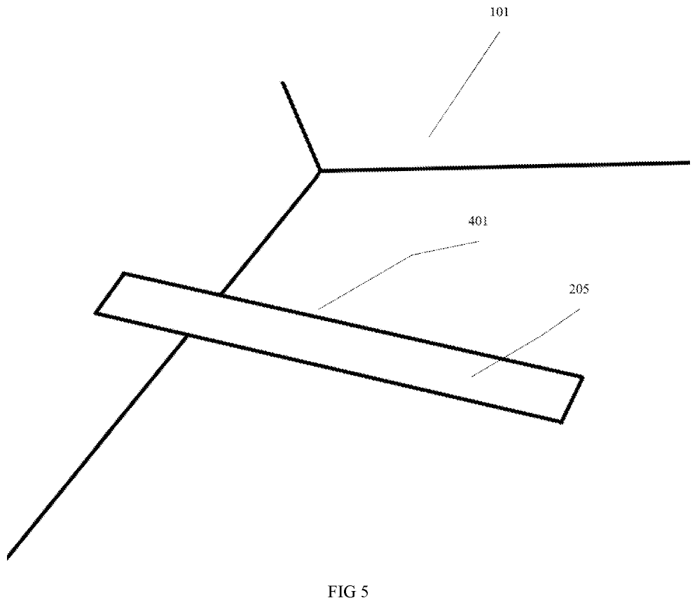

FIG. 6 is a perspective view, which shows an Overview of the Fluid Storage and Pumping.

FIG. 7 is a perspective view, which shows an Overview of the Fluid Supply Side.

FIG. 8 is a perspective view, which shows an Overview of the Fluid Return Side.

FIG. 9 is a perspective view, which shows an Isometric view of the Slider Pipe.

FIG. 10 is a perspective view, which shows an Isometric view of the Use of the Ninety-Degree End Cap.

FIG. 11 is a perspective view, which shows an Isometric view of the Adjustable Valve.

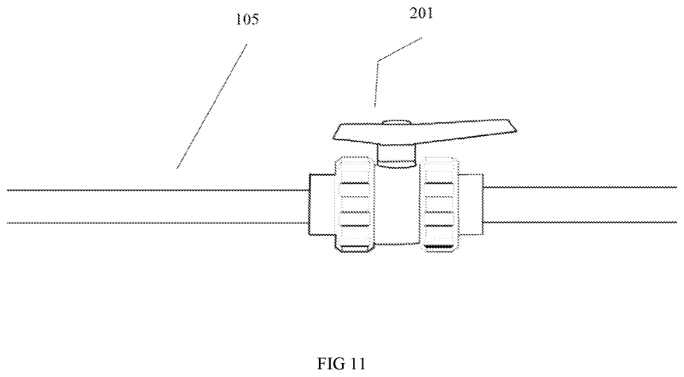

### DETAILED DESCRIPTION

One or more different inventions may be described in the present application. Further, for one or more of the inventions described herein, numerous alternative embodiments may be described; it should be appreciated that these are presented for illustrative purposes only and are not limiting of the inventions contained herein or the claims presented herein in any way. One or more of the inventions may be widely applicable to numerous embodiments, as may be readily apparent from the disclosure. In general, embodiments are described in sufficient detail to enable those skilled in the art to practice one or more of the inventions, and it should be appreciated that other embodiments may be utilized and that structural, logical, software, electrical, and other changes may be made without departing from the scope of the particular inventions. Accordingly, one skilled in the art will recognize that one or more of the inventions may be practiced with various modifications and alterations. Particular features of one or more of the inventions described herein may be described with reference to one or more particular embodiments or figures that form a part of the present disclosure, and in which are shown, by way of illustration, specific embodiments of one or more of the inventions. It should be appreciated, however, that such features are not limited to usage in one or more particular embodiments or figures with reference to which they are described. The present disclosure is neither a literal description of all embodiments of one or more of the inventions nor a listing of features of one or more of the inventions that must be present in all embodiments.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an embodiment with several components in communication with each other does not imply that all such components are required. On the contrary, a variety of optional components may be described to illustrate a wide variety of possible embodiments of one or more of the inventions and in order to more fully illustrate one or more aspects of the inventions. Similarly, although process steps, method steps, algorithms, or the like may be described in sequential order, such processes, methods, and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the invention(s), and does not imply that the illustrated process is preferred. Also, steps are generally described once per embodiment, but this does not mean they must occur once, or that they may only occur once each time a process, method, or algorithm is carried out or executed. Some steps may be omitted in some embodiments or some occurrences, or some steps may be executed more than once in a given embodiment or occurrence.

When a single device or article is described herein, it will be readily apparent that more than one device or article may be used in place of a single device or article. Similarly, where more than one device or article is described herein, it will be readily apparent that a single device or article may be used in place of more than one device or article.

The functionality or the features of a device may be alternatively embodied by one or more other devices that are not explicitly described as having such functionality or features. Thus, other embodiments of one or more of the inventions need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code that include one or more executable instructions for implementing specific logical functions or steps in the process. Alternate implementations are included within the scope of embodiments of the present invention in which, for example, functions may be executed out of order from that shown or discussed, including substantially concurrently or in reverse order, depending on the functionality involved, as would be understood by those having ordinary skill in the art.

As used throughout this application parts which are described as being pipes are defined as things that are capable of transmitting fluids or other fluid substances. As used throughout this application a pipe could also be PVC pipes, copper conduit pipes, metal pipes, rubber pipes, PEX tubing, and the like. As used throughout this invention, the Pipe could have a diameter that ranges from ⅛ inch to ten inches.

The Automated Fluid Delivery System comprises one or more Delivery Destinations (101), a Fluid Delivery System (108), a Fluid Sanitation System (208), and a Pumping and Storage System (106).

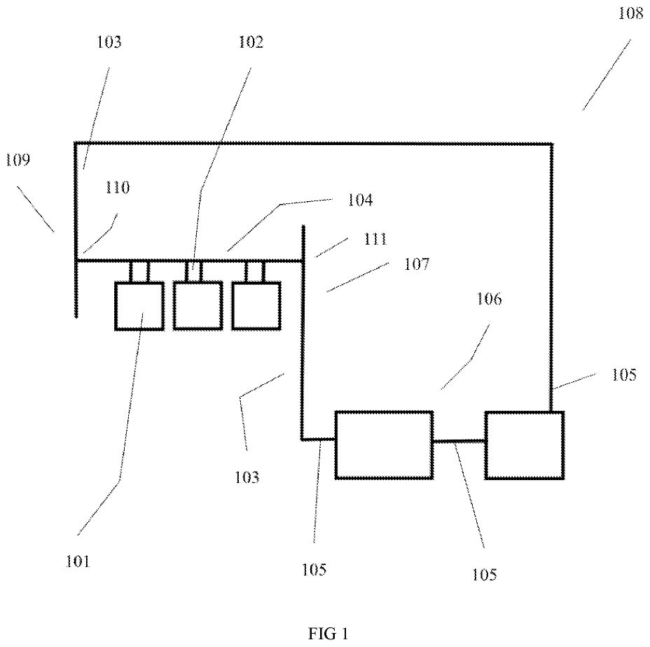

Referring now to FIG. 1, which shows an Overall Perspective view of the System.

The Fluid Delivery System (108) comprises one or more Interconnecting Pipes (105), one or more Continuous Fluid Dispensers (102), one or more Fluid Supply Pipes (104), one or more Fluid Flow Restriction Systems (301), a Fluid Removal System (210), and one or more Branching Connection Pipes (103) and a Delivery Fluid (205). The purpose of the Fluid Delivery System (108) is to transport the Delivery Fluid (205) to the Delivery Destination (101) for consumption and/or use. The Fluid Delivery System (108) operably interacts with the Delivery Fluid (205) and the Pumping and Storage System (106) to transport the Delivery Fluid (205) to the Delivery Destination (101) through the pipes which are part of the Fluid Delivery System (108). The Fluid Delivery System (108) contains pipes that are connected together to form a closed loop and operably works in concert with the Pumping and Storage System (106) to transport the Delivery Fluid (205) from the Pumping and Storage System (106) to the Delivery Destination (101).

The Fluid Supply Pipe (104) is operably connected at both ends to one or more Branching Connection Pipes (103) via one or more Fluid Flow Restriction Systems (301). The Fluid Supply Pipe (104) comprises at least one or more Fresh Fluid Outlet Apertures (901), one or more Slider Pipes (206), a Fluid Supply Pipe Inlet (110) and a Fluid Supply Pipe Outlet (111), and one or more Ninety Degree End Caps (1001). The Fluid Supply Pipe (104) is operably connected via one or more Fresh Fluid Outlet Apertures (901) to one or more Continuous Fluid Dispensers (102). The Fluid Supply Pipe (104) is a type of pipe as defined in this application. The purpose of the Fluid Supply Pipe (104) is to transport the Delivery Fluid (205) to and from one or more Continuous Fluid Dispensers (102). The Fluid Supply Pipe Inlet (110) is operably connected to the Fluid Supply Side (109) via a Fluid Flow Restriction System (301) and a Ninety Degree End Cap (1001). The Ninety Degree End Cap (1001) is preferably located on the side of the Fluid Supply Pipe (104) which is Operably connected to the Fluid Supply Side (109). Fluid Supply Pipe Outlet (111) is operably connected to the Fluid Return Side (107).

The Interconnecting Pipes (105) comprises a Storage Reservoir to Pump Pipe (602), a Main Fluid Return Side to Storage Reservoir Pipe (603), and a Pump to Main Fluid Supply Side Pipe (601). One purpose of the Interconnecting Pipes (105) is to provide for the transport of the Delivery Fluid (205) between the Fluid Supply Pipe (104) and one or more of the Branching Connections Pipes (103). The Interconnecting Pipes (105) are defined as a type of pipe as defined in this application.

The Continuous Fluid Dispenser (102) comprises at least one Fresh Fluid Outlet (401). The purpose of the Continuous Fluid Dispenser (102) is to both transport and deliver the Delivery Fluid (205) for its consumption and use at the Delivery Destination (101). The Continuous Fluid Dispenser (102) transports the Delivery Fluid (205) from the Fluid Supply Pipe (104) to the Delivery Destination (101). The Continuous Fluid Dispenser (102) could be a rope, yarn, or any material capable of wicking the Delivery Fluid (205) through the Continuous Fluid Dispenser (102).

One purpose of the Delivery Destination (101) is to act as the Delivery Destination (101) of the Delivery Fluid (205). The Delivery Destination (101) is operably attached to the Pumping and Storage System (106) and the Fluid Delivery System (108). If the Delivery Destination (101) is absent, then there is no place for the Delivery Fluid (205) to be transported to. The Delivery Destination (101) could also be a Box.

The Pumping and Storage System (106) comprises one or more Storage Reservoirs (203), one or more Sanitizing Filters (211), and one or more Pumps (202). If the Pumping and Storage System (106) is absent, then the Delivery Fluid (205) is unable to move throughout the Fluid Delivery System (108). The Pumping and Storage System (106) is operably connected to the Fluid Delivery System (108). The Pumping and Storage System (106) operably interacts to move the Delivery Fluid (205) through the Fluid Delivery System (108).

In one embodiment of the invention, the Pumping and Storage System (106) comprises at least two Storage Reservoirs (203). In this embodiment of the invention, at least one Storage Reservoir (203) is positioned at an overall elevation that is lower than the relative elevation of the outlet of the Fluid Delivery System (108). At least one Storage Reservoir (203) is located at a relative elevation that is higher than the inlet of the Fluid Delivery System (108). Further in this embodiment, at least one Storage Reservoir (203) that is located below the Fluid Delivery System (108) is operably connected via one or more pipes to a Pump (202) which is then operably connected via one or more pipes to a Storage Reservoir (203) located at an elevation that is higher than the inlet of the Storage Reservoir (203). In this embodiment, the Pump (202) serves to operably transfer the Delivery Fluid (205) from the lower Storage Reservoir to one or more Storage Reservoirs (203) which are positioned at the higher elevation. The Delivery Fluid (205) is then permitted to transverse the Fluid Delivery System (108) under the effects of gravity and ultimately return to one or more Storage Reservoirs (203) positioned at a relative elevation which is lower than the elevation of the Fluid Delivery System (108). In one embodiment of the invention Delivery Fluid (205) passes through the Sanitizing Filter (211) on its way to one of the Storage Reservoirs (203) which are located at the higher elevation. This Sanitizing Filter (211) can also be a UV Light filter which serves to kill any bacteria and viruses present in the Delivery Fluid. (205) In another embodiment of the invention the Sanitizing Filter is located down pressure from one or more Storage Reservoirs (203).

The Fluid Supply Side (109) comprises one or more Main Fluid Supply Side Pipes (701). The Fluid Supply Side (109) operably connects to the Pumping and Storage System (106) via one or more Interconnecting Pipes (105). The Fluid Supply Side (109) is positioned within the Fluid Delivery System (108) at a location within the closed-loop of the Fluid Delivery System (108) down pressure from the Pumping and Storage System (106) but up pressure from the inlet of one or more Fluid Supply Pipes (104). The Fluid Supply Side (109) is positioned up-pressure from one or more Delivery Destinations (101).

The Fluid Return Side (107) comprises one or more Main Fluid Return Side Pipes (801). The Fluid Return Side (107) operably connects to the Storage Reservoir (203) via one or more Interconnecting Pipes (105). The Fluid Return Side (107) is positioned within the Fluid Delivery System (108) at a location within the closed-loop of the Fluid Delivery System (108) prior to the inlet of the Pumping and Storage System (106) and down pressure from an outlet of one or more Fluid Supply Pipes (104).

The Branching Connection Pipe (103) is operably connected to one or more Interconnecting Pipes (105). The Branching Connection Pipe (103) is operably connected to the Fluid Supply Pipe (104) via the Fluid Flow Restriction System (301). The Branching Connection Pipe (103) comprises the Fluid Supply Side (109) and the Fluid Return Side (107).

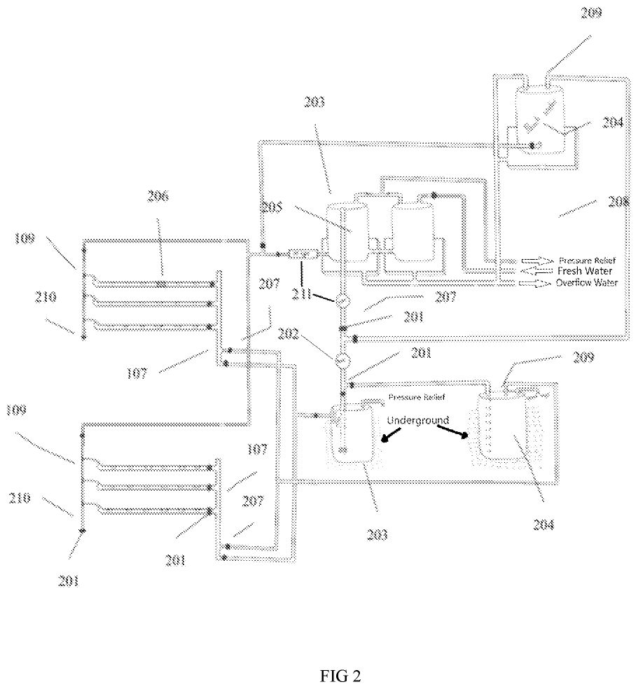

Referring now to FIG. 2, which shows Overview of Entire System Including the Fluid Sanitation System (208).

The Delivery Fluid (205) is defined as a fluid that is capable of wicking up and through the Continuous Fluid Dispenser (102) and is also capable of transmission through the Fluid Delivery System (108) via the Pumping and Storage System (106). In a preferred embodiment of the invention, the Delivery Fluid (205) is thought to be water. In a preferred embodiment of the invention, it is contemplated that the Delivery Fluid (205) has the property where its adhesion force is greater than its cohesive force. It is also contemplated that the Delivery Fluid (205) could be any substance capable of showing capillary action. However, the Delivery Fluid (205) could also be any water-based substance, mineral oil, alcohol, gasoline, and the like. In a preferred embodiment of the invention, the Delivery Fluid (205) has a contact angle that is less than ninety degrees.

The Fluid Sanitation System (208) comprises a Fluid Redirection Network (207), one or more Pumps (202), one or more Sanitation Storage Reservoirs (209), and Sanitation Fluid (204). The purpose of the Fluid Sanitation System (208) is to sanitize the various pipes of the Fluid Delivery System (108) by using the Fluid Redirection Network (207) to divert the flow of fluid from the Storage Reservoir (203) to the Sanitation Storage Reservoir (209). Then the Sanitation Fluid (204) is allowed to remain in the Fluid Delivery System (108) for a period of time. In a preferred embodiment of the invention, this period of time is at least two hours or until all the bleach in the Sanitation Fluid (204) has disintegrated. The Fluid. Delivery System (108) is then flushed out with Delivery Fluid (203) so that the Fluid Delivery System (108) is once again ready for the transport of the Delivery Fluid (205).

The Fluid Redirection Network (207) comprises one or more Adjustable Valves (201) which are operably connected to the Fluid Supply Side (109) and the Fluid Return Side (107). The Purpose of the Fluid Redirection Network (207) is to redirect to the flow of either a Delivery Fluid (205) or Sanitation Fluid (204) between the Fluid Sanitation System (208), the Pumping and Storage System (106), and the Fluid Delivery System (108). In a preferred embodiment of the invention, the Adjustable Valves (201) of the Fluid Redirection Network (207) have at least two positions, in one position the flow of fluid is such that it is directed towards the Storage Reservoir (203). When the position of one or more of these valves is changed, the flow to the Storage Reservoir (203) is stopped; While the fluid is still permitted to flow through one or more Fluid Supply Side (109) and one or more Fluid Return Side (107), the flow through the Storage Reservoir (203) is blocked. In this manner, the flow is redirected through the Fluid Sanitation System (208). When the valves are returned to their other position, the flow of fluid through the Fluid Sanitation System (208) is blocked, and the Delivery Fluid (205) is able to again flow through the Storage Reservoir (203) as well as the newly sanitized and cleaned Fluid Delivery System (108).

The Sanitation Reservoir (209) is defined as a container that can be used to store and contain the Sanitation Fluid (204).

The Sanitation Fluid (204) is a fluid that is capable of cleaning and disinfecting the interior of the pipes of the invention. In a preferred embodiment of the invention, the Sanitation Fluid (204) is comprised of 200 ppm (parts per million) of chlorine bleach as recommended by the Department of Health (DOH) for sanitizing purposes. It is recommended that the concentration of bleach be between 50 and 200 ppm.

The Pump (202) is defined as a device that moves fluids (liquids or gases), or sometimes slurries, by mechanical action, typically converted from electrical energy into hydraulic energy.

The purpose of the Fluid Removal System (210) is to provide an opening in the system whereby the closed-loop of the Fluid Delivery System (108) can be opened and allow for the removal of the Delivery Fluid (205) and/or Sanitation Fluid (204) from the system at a location other than the Storage Reservoir (203). The Fluid Removal System (210) could also be an Adjustable Valve (201), a cap on the pipe, or the like.

The Storage Reservoir (203) is defined as a container that can be used to store and contain the Delivery Fluid (205). In one embodiment of the invention the Storage Reservoir (203) contains a valve that shuts off the Pump (202) and stops the flow of Delivery Fluid (205) and/or Sanitation Fluid (204) when either the Delivery Fluid (205 and/or Sanitation Fluid (204) reaches a certain level within one or more Storage Reservoirs (203).

The Slider Pipe (206) is defined as a pipe having a cross-sectional area that is larger than that of a Fluid Supply Pipe (104). The purpose of the Slider Pipe (206) is to operably traverse the exterior face of the Fluid Supply Pipe (104). When placed in certain positions along the exterior face of the Fluid Supply Pipe (104) the Slider Pipe (206) covers one or more of Fresh Fluid Outlet Aperture (901). The purpose of this covering is to provide a mechanism whereby these apertures can be conveniently closed when one or more Continuous Fluid Dispensers (102) are absent. The Slider Pipe (206) preferably has a length that can be defined based on the dimensions between the outside edges of at least one Fresh Fluid Outlet Aperture (901). In a preferred embodiment of the invention, the Slider Pipe (206) has a length sufficient to cover the Fresh Fluid Outlet Aperture (901) completely.

The Adjustable Valve (201) is a device or natural object that regulates, directs, or controls the flow of a fluid (gases, liquids, fluidized solids, or slurries) by opening, closing, or partially obstructing various passageways. As used in this invention, the purpose of the Adjustable Valve (201) is to allow for the setting of the position of the valve in a partially opened position such that the effective cross-sectional area of the Fluid Flow Restriction System (301) is maintained between 5% and 99%. Another purpose of the Adjustable Valve (201) is to redirect and determine whether the Delivery Fluid (205) and/or Sanitation Fluid (204) are permitted to traverse the Fluid Delivery System (108).

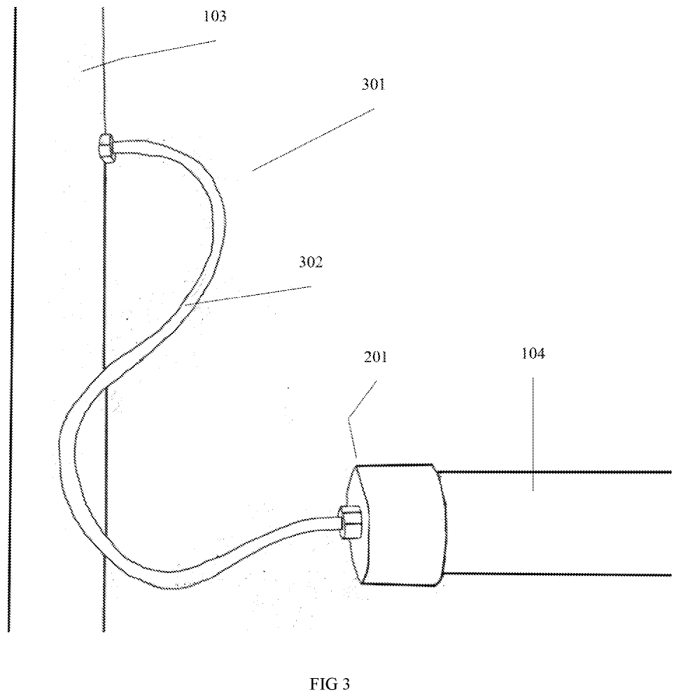

Referring now to FIG. 3, which shows Perspective View of the Fluid Flow Restriction System (301).

The purpose of the Fluid Flow Restriction System (301) is to ensure that there is an increased pressure and fluid level in the Fluid Supply Pipe (104) compared to the pressure and fluid level in the Branching Connection Pipe (103). Another purpose of the Fluid Flow Restriction System (301) is to provide a mechanism whereby the diameter of the cross-sectional area of the inlet and exit of the Fluid Supply Pipe (104) is reduced. If the Fluid Flow Restriction System (301) is absent, then the pressure and fluid level of the Fluid Supply Pipe (104) would be the same as the fluid pressure and level in the Branching Connection Pipe (103). The purpose of maintaining this higher pressure is to ensure that there is always a continuous flow of fluid through the Continuous Fluid Dispenser (102). The Fluid Flow Restriction System (301) comprises either a Restriction Pipe (302) or an Adjustable Valve (201). If the Restriction Pipe (302) is absent, then the pressure is maintained via the Adjustable Valve (201) alone. If the Adjustable Valve (201) is absent, then the pressure is maintained via the Restriction Pipe (302) alone. The Fluid Flow Restriction System (301) has a preferred reducing diameter that can be calculated as a diameter that is less than that of the Branching Connection Pipe (103) to which it is operably connected to. In a preferred embodiment of the invention, the Fluid Flow Restriction System (301) has a preferred reducing diameter that is ¼ of an inch in diameter. In another embodiment of the invention, the Fluid Flow Restriction System (301) has a diameter that is between 3% and 99% of the diameter of the inlet of the Fluid Supply Pipe (104).

The Restriction Pipe (302) is a pipe whose cross-sectional area can be calculated as a percentage of the cross-sectional area of the Fluid Supply Pipe (104). The cross-sectional area of the Restriction Pipe (302) is less than that of the Fluid Supply Pipe (104) to which it is operably connected. In a preferred embodiment of the invention, the Restriction Pipe (302) has a cross-sectional area that is 97% of the cross-sectional area of the Fluid Supply Pipe (104) to which it is operably connected. However, it is contemplated that the Restriction Pipe (302) has a cross-section area that can range from between 3% and 99% of the cross-sectional area of the Fluid Supply Pipe (104) to which it is operably connected.

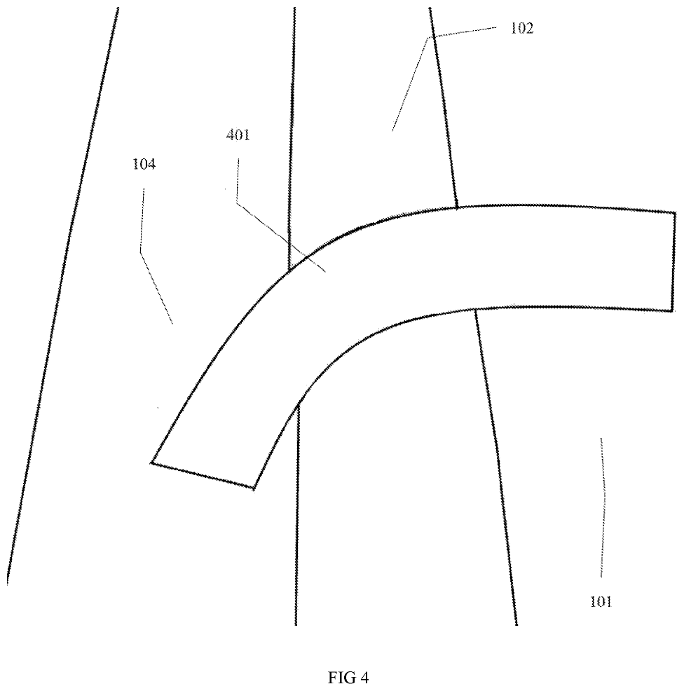

Referring now to FIG. 4, which shows Perspective View of Continuous Fluid Dispenser (102).

The Fresh Fluid Outlet (401) is defined as the section of the Continuous Fluid Dispenser (102) that is located on the outlet side prior to the Delivery Fluid (205) has been transported through the Delivery Destination (101). The Fresh Fluid Outlet (401) is the mechanism by which the Delivery Fluid (205) travels into the Delivery Destination (101). In a preferred embodiment of the invention, the Fresh Fluid Outlet (401) is a rope. In a preferred embodiment of the invention, the Fresh Fluid Outlet (401) is composed of a material that is capable of wicking the Delivery Fluid (205).

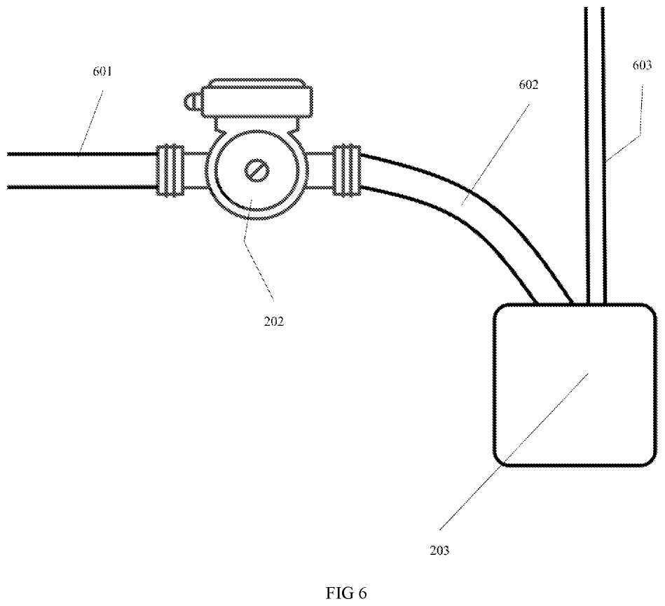

Referring now to FIG. 6, which shows Overview of Fluid Storage and Pumping.

The Pump to Main Fluid Supply Side Pipe (601) is a transporting pipe that is operably connected to the Pump (202) and Fluid Supply Side (109). The purpose of the Pump to Main Fluid Supply Side Pipe (601) is to provide for the delivery of the Delivery Fluid (205) from the Pump (202) to the Fluid Supply Side (109). The Main Fluid Return Side to Storage Reservoir Pipe (603) is defined as a type of pipe as defined in this application.

The Storage Reservoir to Pump Pipe (602) is a transporting pipe that is operably connected to the Storage Reservoir (203) at one end and at the other end is operably connected to the Pump (202). The purpose of the Storage Reservoir to Pump Pipe (602) is to provide for transport between the Storage Reservoir (203) and the Pump (202). The Storage Reservoir to Pump Pipe (602) is defined as a type of pipe as defined in this application.

The Main Fluid Return Side to Storage Reservoir Pipe (603) is a transporting pipe that is operably connected to the Storage Reservoir (203) and the Fluid Return Side (107). The purpose of the Main Fluid Return Side to Storage Reservoir Pipe (603) is to provide for the return of the Delivery Fluid (205) to the Storage Reservoir (203). The Main Fluid Return Side to Storage Reservoir Pipe (603) is defined as a type of pipe as defined in this application.

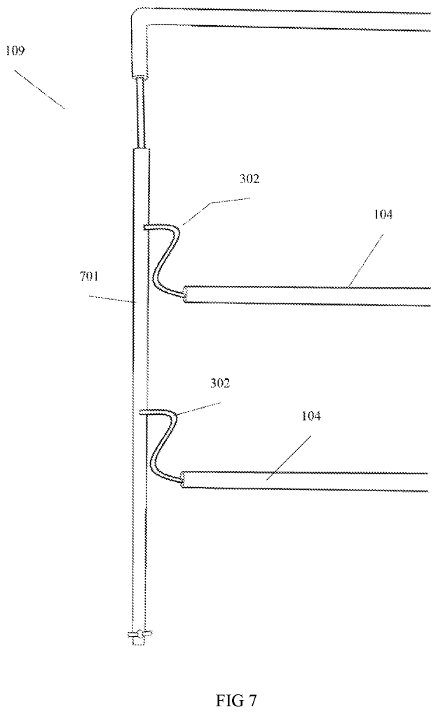

Referring now to FIG. 7, which shows Overview of the Fluid Supply Side (109).

The Main Fluid Supply Side Pipe (701) is defined as a pipe as the term is used and understood in this application.

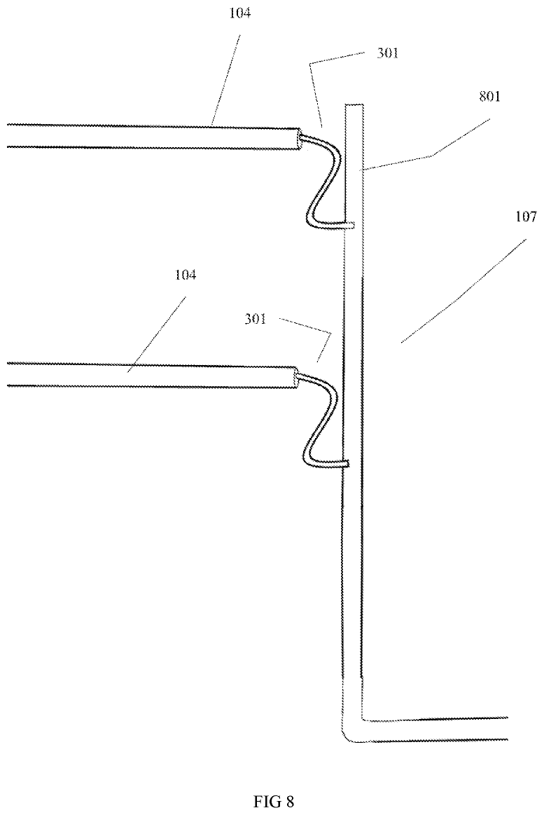

Referring now to FIG. 8, which shows an Overview of the Fluid Return Side (107).

The Main Fluid Return Side Pipe (801) is defined as a pipe as defined in this application.

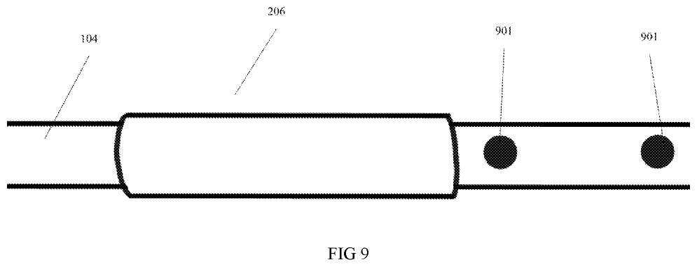

Referring now to FIG. 9, which shows an Isometric view of the Slider Pipe (206).

The Fresh Fluid Outlet Aperture (901) is an aperture located on the Fluid Supply Pipe (104) whose purpose is to allow for the operable connection of the outlet of the Continuous Fluid Dispenser (102).

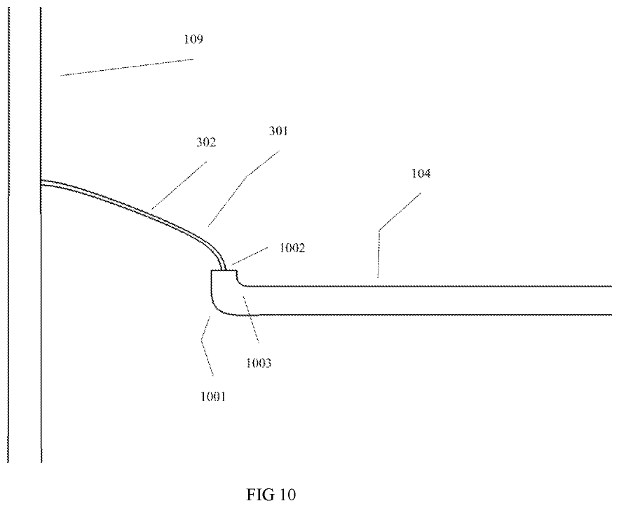

Referring now to FIG. 10, which shows Isometric view of the Use of the Ninety-Degree End Cap.

The Ninety Degree End Cap (1001) is defined as a pipe that comprises a Ninety Degree End Cap Inlet (1002) and a Ninety Degree End Cap Outlet (1003) in which the exterior face of the Ninety Degree End Cap Inlet (1002) and the exterior face of the Ninety Degree End Cap Outlet (1003) are aligned on planes which are perpendicular to each other. The purpose of the Ninety Degree End Cap (1001) is to provide a bend in the entrance of the Fluid Supply Pipe (104) which provides a physical barrier preventing the egress of Delivery Fluid (205) back towards the Fluid Supply Side (109). In a preferred embodiment of the invention, the sizing of the Ninety Degree End Cap Inlet (1002) and the Ninety Degree End Cap Outlet (1003) are relative such that the diameter of the Ninety Degree End Cap Outlet (1003) is larger than the diameter of the Ninety Degree End Cap inlet (1002).

What is claimed is:

1. An apparatus comprising:
a. One or more Delivery Destinations
b. a Fluid Delivery System;
c. a Fluid Sanitation System; and
d. a Pumping and Storage System which is operably attached to the Fluid Delivery System and one or more of the Delivery Destinations.

2. The apparatus of claim 1 wherein the Fluid Delivery System further comprises:
a. one or more Interconnecting Pipes;
b. one or more Continuous Fluid Dispensers;
c. one or more Fluid Supply Pipes;
d. one or more Fluid Flow Restriction Systems;
e. a Fluid Removal System;
f. one or more Branching Connection Pipes; and
g. a Delivery Fluid.

3. The apparatus of claim 2 wherein the pumping and Storage System further comprises:
a. one or more Storage Reservoirs;
b. one or more Sanitizing Filter; and
c. and one or more Pumps.

4. The apparatus of claim 3 wherein the Fluid Sanitation System further comprises:
a. a Fluid Redirection Network;
b. one or more Sanitation Storage Reservoirs; and
c. a Sanitation Fluid.

5. The apparatus of claim 4 wherein the Pumping and Storage System transports the Delivery Fluid through the Fluid Delivery system and ends up at the Delivery destination.

6. The apparatus of claim 3 wherein the Pumping and Storage System comprises two or more Storage Reservoirs.

7. The apparatus of claim 6 wherein at least one of the Storage Reservoirs is positioned at an overall elevation that is higher than the inlet of the Fluid Delivery System.

8. An apparatus comprising:
a. a Fluid Delivery System wherein the Fluid Delivery System further comprises a Fluid Flow Restriction System;
b. a Pumping and Storage System wherein the Pumping and Storage System further comprises:
i. one or more Storage Reservoirs;
ii. one or more Sanitizing Filters; and
iii. one or more Pumps;
c. one or more Delivery Destinations; and
d. a Fluid Sanitation System wherein the Fluid Sanitation System further comprises
i. one or more Pumps;
ii. a Fluid Redirection Network;
iii. one or more Sanitation Storage Reservoirs; and
iv. a Sanitation Fluid.

9. The apparatus of claim 8 wherein the Fluid Delivery System further comprises:
a. one or more Interconnecting Pipes;
b. a Continuous Fluid Dispensers;
c. one or more Branching Connection Pipes;
d. a Fluid Removal System System;
e. a Delivery Fluid; and
f. one or more Fluid Supply Pipes.

10. The apparatus of claim 9 wherein the Fluid Supply pipes further comprises a Fluid Supply Side and a Fluid Return side wherein the Fluid Redirection Network further comprises one or more adjustable valves which are operably connected to the inlet of the Fluid Supply Side.

11. The apparatus of claim 10 wherein the Adjustable Valves have at least two positions, in one position the flow to the Storage Reservoir is stopped; however in this position the Sanitation Fluid is permitted to flow through one the Fluid Supply Side and the Fluid Return Side, the flow through the Storage Reservoir is blocked and the Sanitation Fluid is directed through the Fluid Sanitation System, this is contrasted from when the valves are returned to their other position, the flow of fluid through the Fluid Sanitation System is blocked, and the Delivery Fluid is able to flow through the Storage Reservoir as well as the newly sanitized and cleaned Fluid Delivery System.
